(19) United States
(12) Patent Application Publication
(10) Pub. No.: US 2025/0002952 A1
(43) Pub. Date: Jan. 2, 2025

(54) METHOD FOR PREPARING L-CYSTEIC ACID

(71) Applicant: WACKER CHEMIE AG, Munich (DE)

(72) Inventors: Rupert PFALLER, Munich (DE); Thomas SCHLÖSSER, Burghausen (DE); Günter WICH, Munich (DE)

(73) Assignee: WACKER CHEMIE AG, Munich (DE)

(21) Appl. No.: 18/709,817

(22) PCT Filed: Nov. 29, 2021
(86) PCT No.: PCT/EP2021/083372

§ 371 Date: May 14, 2024

(51) Int. Cl. C12P 13/12; C12N 9/88

(52) U.S. Cl. CPC C12P 13/12; C12N 9/88; C12Y 402/99

(57) ABSTRACT

Methods and processes for preparing L-cysteic acid. Where according to the process, acetyl-L-serine (OAS) is converted using at least one enzyme selected from the class of O-acetyl-L-serine sulfhydrylases (OAS sulfhydrylases, EC 4.2.99.8) in the presence of a salt of sulfurous acid. Where the OAS sulfhydrylase is CysM and the biotransformation is carried out under active pH control and the OAS concentration in the batch is at least 10 g/L.

The invention relates to a process for producing L-cysteic acid, wherein O-acetyl-L-serine (OAS) is converted using at least one enzyme selected from the class of O-acetyl-L-serine sulfhydrylases (OAS sulfhydrylases, EC 4.2.99.8) in the presence of a salt of sulfurous acid. L-cysteic acid is provided as a result of this biotransformation.

L-cysteic acid can be used, for example, in fish farming (Nakamura et al., Fisheries Science (2021) 87:353-363) or in the cosmetics sector (U.S. Pat. No. 4,053,630), for example as an ingredient of Regu®-Slim (DSM) for skin care. In peptide chemistry, L-cysteic acid is used as a water-soluble protective group. Furthermore, L-cysteic acid can be converted to taurine by decarboxylation.

L-cysteic acid ((R)-2-amino-3-sulfopropanoic acid, 3-sulfo-L-alanine, CAS 498-40-8) can be produced chemically, for example by oxidation of cysteine with chlorine in an alcoholic solution (Tao et al. Amino Acids (2004) 27:149-151), with bromine in HCl or iodine HCl in DMSO, or by oxidative cleavage of cystine with performic acid. Furthermore, L-cysteic acid can also be produced by oxidation of L-cysteine sulfinic acid. The known processes for chemical production of L-cysteic acid, which are not considered sustainable, use environmentally hazardous chemicals and have low consumer acceptance, especially in applications in the food, cosmetics and pharmaceutical sectors. There is therefore a need for a more environmentally friendly and more sustainable production process, one option for which is a biotechnological process.

L-cysteic acid is a non-proteinogenic L-amino acid that can be detected in nature as an oxidation product of the proteinogenic amino acid L-cysteine, for example in sheep's wool. Cysteic acid is also an intermediate of coenzyme M (COM, 2-mercaptoethanesulfonic acid, CAS 3375-50-6) biosynthesis by methanogenic archaebacteria.

The prior art provides processes for producing non-proteinogenic amino acids, for example by direct fermentation of microorganisms with deregulated cysteine metabolism (EP 1 191 106 B1) or by OAS sulfhydrylase-catalyzed biotransformation of OAS (EP 1 247 869 B1). The processes are based on an OAS sulfhydrylase catalyzing the reaction of OAS with a nucleophile to form a non-proteinogenic amino acid according to the general formula (1):

OAS+Nucleophile->Non-proteinogenic amino acid+Acetate (1)

In the cysteine metabolism of, for example, Escherichia coli, OAS serves as a biosynthetic precursor of L-cysteine. The latter is formed by substitution of the acetate group at the beta position by a thiol radical. This reaction, referred to as beta-substitution, is catalyzed by enzymes of the class of OAS sulfhydrylases (EC 4.2.99.8). Therefore, OAS is the actual substrate (also referred to as reactant) of the OAS sulfhydrylase reaction and the nucleophile is the variable cosubstrate.

In EP 1 247 869 B1, a multitude of different nucleophiles were tested for their suitability as nucleophile for the OAS sulfhydrylase-catalyzed (e.g., CysM-catalyzed) reaction with OAS, including selenides, selenol, azides, cyanides, azoles and isoxazolinones. In addition, sulfur compounds from the group of thiosulfates and thiols of the general formula H—S—R, the radical R being a monovalent substituted or unsubstituted alkyl, alkoxy, aryl or heteroaryl radical, were tested.

What were produced were non-proteinogenic amino acids such as S-phenyl-L-cysteine, which are not used in nature as building blocks for protein biosynthesis. None of the nucleophiles disclosed allows the production of L-cysteic acid.

Joo et al. (2018), J. Agric. Food Chem. 66:13454-13463, describe a metabolic engineering approach for the production of taurine in the bacterium Corynebacterium glutamicum. The genes of an L-cysteine synthase, a cysteine dioxygenase and an L-cysteine sulfinic acid decarboxylase were heterologously expressed in the strain in order to arrive at the production of taurine. FIG. 2 of Joo et al. (2018), J. Agric. Food Chem. 66:13454-13463, describes various metabolic pathways to taurine, these also including a pathway (“L-cysteine sulfonic acid pathway”) which starts from O-phospho-L-serine and leads to taurine via L-cysteic acid and which would in principle also be suitable for production of L-cysteic acid. However, the figure also shows that there is no known biosynthesis pathway leading from OAS to L-cysteic acid, only to L-cysteine.

Tevatia et al., Algal Research (2015) 9:21-26, describe the natural production of taurine in microalgae, with L-cysteic acid also being detected as an intermediate. As described in FIG. 1a) of Tevatia et al., Algal Research (2015) 9:21-26, a biosynthesis pathway leads from L-serine to L-cysteic acid (“Cysteate” in FIG. 1a). None of the biosynthesis pathways described leads to L-cysteic acid via OAS. The intracellular content of L-cysteic acid detected in microalgae was very low and was accompanied by multiple by-products that make workup more difficult, such as methionine, cysteine, cysteine sulfinic acid, hypotaurine and taurine, and so the growth of microalgae is unsuitable for the production of L-cysteic acid.

In a metabolic engineering approach, US 2019/0062757 A1 (KnipBio) describes heterologous production strains for the production of taurine, said strains also being intended to be suitable for production of L-cysteic acid. FIGS. 4 to 9 and 12 of US 2019/0062757 A1 describe various biosynthesis pathways to taurine that contain L-cysteic acid as an intermediate and would therefore in principle be suitable for production of L-cysteic acid. None of these biosynthesis pathways starts from OAS. Furthermore, only hypotaurine and taurine yields were reported and they were very low with a maximum of 419 ng/ml. Yields for the production of L-cysteic acid were not mentioned. It must be assumed that higher yields cannot be achieved for L-cysteic acid. This metabolic engineering approach is therefore unsuitable for the biotechnological production of L-cysteic acid.

The prior art therefore only discloses chemical processes and does not disclose an economically viable biotechnology process for producing L-cysteic acid that would be suitable for industrial use.

It is an object of the present invention to provide a biotechnological process for producing L-cysteic acid by biotransformation.

The object is achieved by a process for producing L-cysteic acid, wherein O-acetyl-L-serine (OAS) is converted using at least one enzyme selected the from class of O-acetyl-L-serine sulfhydrylases (OAS sulfhydrylases, EC 4.2.99.8) in the presence of a salt of sulfurous acid. This process provides L-cysteic acid produced by biotransformation.

The advantage of the process according to the invention is that it is a sustainable and technically feasible biotransformation process for producing L-cysteic acid. Environmentally hazardous chemicals can be dispensed with. Fossil raw materials are not consumed and toxic chemical waste and/or waste gases are not produced. The production process of the present invention is therefore environmentally friendly and sustainable. Moreover, the process does not require either extreme reaction conditions or special equipment and is therefore easy to technically implement. Another advantage of the process is that natural L-cysteic acid, which is increasingly in demand, can be produced in this way.

It has been found that, surprisingly, salts of sulfurous acid (referred to hereinafter as sulfites or SO₃²⁻) are suitable as nucleophile in reaction (1) and allow the synthesis of L-cysteic acid in a hitherto unknown reaction according to equation (2).

OAS+SO₃²⁻->L-Cysteic acid+Acetate (2)

In the context of the present invention, production processes are distinguished as follows:
1. Chemical processes
2. Biotechnological processes:
   a) by metabolic engineering
      Metabolic engineering (also called “pathway design”), in contrast to biotransformation, is a biotechnology method in which metabolic pathways of an organism are modified by optimization or modification of genetic and regulatory processes. New or modified enzymes can be introduced into an organism by supplementation of the genome with genes of enzymes, or genes of endogenous enzymes can be expressed at an enhanced or attenuated level, thereby establishing new metabolic pathways in an organism or enhancing or attenuating existing metabolic pathways. The goal of metabolic engineering is for the organism to produce either a new metabolite or a cell-endogenous metabolite at increased yield. A metabolic engineering process does not use starting materials specific for the metabolite, such as an enzyme substrate, for example OAS in the present invention; instead, it only uses a nutrient medium, also referred to as growth medium, which is required for the growth of the organism in question and is composed of a carbon source (e.g., glucose), a nitrogen source (e.g., an ammonium salt or a complex amino acid mixture such as peptone or yeast extract) and other salts required for growth. Such nutrient media are known to a person skilled in the art from microbiological practice.
   b) by biotransformation
      Biotransformation is defined as the transformation of one or more reactants into a product under enzymatic catalysis, the enzyme substrate being added with the enzyme to a reaction batch. In the reaction batch, the enzyme substrate added, such as OAS in the present invention, is converted enzymatically (in the present invention, by an enzyme selected from the class of OAS sulfhydrylases (EC 4.2.99.8) in the presence of a salt of sulfurous acid) according to equation (2). The reactant(s) can stem from chemical or biotechnological production. The OAS used in the process according to the invention can stem, for example, from chemical synthesis or from biotechnological production by fermentation of a production strain. The enzyme used for enzymatic catalysis stems from biotechnological production by growth of a production strain, for example by fermentation, or biological material containing the enzyme is used (e.g., plants, fungi, algae, animal organs). The biomass from the growth of the production strain, or the biological material, can be used directly, or the enzyme is isolated therefrom depending on the requirements of the biotransformation. The CysM enzyme used in the process according to the invention stems from biotechnological production by fermentation of a production strain.

A natural production process is defined as a biotechnological production process which does not use genetically modified organisms (GMOs) or products (reactants, enzymes) from production using GMOs. In the present invention, the production process for L-cysteic acid is a natural production process when OAS as organic reactant and the OAS sulfhydrylase have not been produced using GMOs and have not been produced chemically. Sulfite as cosubstrate in equation (2) is an inorganic compound and is fundamentally the product of dissolution of SO₂ in water according to equations (4) to (8), which does not correspond to an (irreversible) chemical synthesis, but to the reversible hydration of the gas SO₂ and the pH-dependent dissociation of the hydrate H₂SO₃.

Self-cloning within the meaning of § 3 No. 3 sentence 4 of the Gentechnikgesetz (GenTG, German Genetic Engineering Act) is, according to a statement (reference: 6790-10-2 of 1991) issued by the Zentrale Kommission für biologische Sicherheit (ZKBS, German Central Committee on Biological Safety), a process in which genetically identical or different forms of only one species, including its viruses and plasmids, are used as donor and recipient organisms.

In the context of the present invention, a reaction batch is defined as a mixture of reactant (starting material), enzyme and optionally other reactants, in which the reactant is converted into a product.

The yield of the reaction within the meaning of the invention is defined as the amount of reactant used that is converted into the product under reaction conditions. The yield can be expressed in terms of absolute amount (g or mmol), as a volume yield (concentration) in terms of absolute amount of product per unit volume (mM or g/L) or as a relative yield of product as a percentage of reactant used (taking into account the molecular weights of the reactant and of the product), also referred to as percent yield.

Fermentation is a process step for the production (cultivation) of cell cultures on an industrial scale, in which a preferably microbial production strain is made to grow under defined conditions of culture medium, temperature, pH, oxygen supply, and mixing of the medium. Depending on the configuration (genetic makeup) of the production strain, the aim of fermentation is to produce a protein/enzyme or a metabolite, in each case in the highest possible yield for further use. The components of the process according to the invention, OAS and OAS sulfhydrylase, can be produced by fermentation. The final of product fermentation is a fermenter broth consisting of the biomass of the cells of the production strain (fermenter cells) and the fermentation medium (fermentation supernatant) which has been removed from the biomass and which has formed during fermentation from the growth medium and the metabolites secreted by the fermenter cells. The target products of fermentation can be present in the fermenter cells or in the fermentation medium. For instance, OAS is found the fermentation medium, whereas the enzyme OAS sulfhydrylase is found in the fermenter cells.

Open reading frame (ORF, synonymous with cds or coding sequence) refers to a region of DNA or RNA that begins with a start codon and ends with a stop codon and encodes the amino acid sequence of a protein. The ORF is also referred to as the coding region or structural gene.

Gene refers to the section of DNA that contains all the basic information for producing a biologically active RNA. A gene contains the section of DNA from which a single-stranded RNA copy is produced by transcription and also the expression signals involved in the regulation of this copying process. The expression signals include for example at least one promoter, a transcription start, a translation start, and a ribosome binding site (RBS). A terminator and one or more operators are additional possible expression signals.

A gene construct refers to a DNA molecule in which a gene is linked to other genetic elements (e.g., promoter, terminator, selection marker, origin of replication). A gene construct in the context of the invention is a circular DNA molecule and is referred to as a plasmid, vector or expression vector. The genetic elements of the gene construct give rise to the extrachromosomal inheritance thereof during cell growth and to the production of the protein encoded by the gene.

L-cysteic acid from the biotransformation of OAS with a salt of sulfurous acid, in accordance with the invention, can be either used further directly without further workup steps or enriched or purified by means of known methods. The degree of enrichment here depends on the further use. Such methods are known to a person skilled in the art from methods for isolating amino acids. Examples include filtration, centrifugation, extraction, adsorption, ion-exchange chromatography, precipitation, crystallization.

In a preferred embodiment, the process is characterized in that L-cysteic acid is enriched from the reaction batch. Particular preference is given to the removal of particulate biomass, for example by centrifugation.

In a further preferred embodiment, the process is characterized in that the L-cysteic acid produced in the process according to the invention is used further directly, i.e., the reaction batch containing L-cysteic acid is used further without: further workup, purification or isolation steps, including filtration, centrifugation, extraction, adsorption, ion exchange chromatography, precipitation and crystallization.

OAS sulfhydrylases have hitherto been isolated from a wide variety of plants and microorganisms. In E. coli, there are for example two OAS sulfhydrylase enzymes, which are referred to as CysK and CysM. The associated genes are likewise known and are designated cysK and cysM, respectively.

OAS sulfhydrylases within the meaning of the present invention are characterized in that they can catalyze the synthesis of the proteinogenic amino acid L-cysteine from OAS according to equation (3), the nucleophile used in this case being sulfide. Both CysM-related and CysK-related enzymes are therefore OAS sulfhydrylases within the meaning of the present invention.

OAS+S²⁻->L-Cysteine+Acetate (3)

Although both enzymes have a very similar reaction mechanism and are involved in the biosynthesis of L-cysteine, CysM, unlike CysK, has a variable substrate spectrum with regard to the nucleophile which can react with OAS according to equation (1).

For example, it is known that CysM, unlike CysK, is capable of catalyzing reaction of OAS with thiosulfate to form S-sulfocysteine (CAS number 1637-71-4). This reaction plays an important role in bacterial growth with thiosulfate as the only source of sulfur.

Furthermore, EP 1 247 869 B1 (Wacker) discloses the use of CysM for production of non-proteinogenic amino acids.

Preferably, the process is characterized in that the OAS sulfhydrylase is a bacterial enzyme, particularly preferably CysM and especially preferably CysM from the strain E. coli.

Sulfurous acid forms a multitude of chemical species simultaneously present in reversible equilibria, the respective suitability of which as nucleophile in the biotransformation according to the invention was not foreseeable. It is thus known that sulfurous acid (H₂SO₃) is the aqueous solution of gaseous SO₂ and is, as a dibasic acid, present in different equilibria depending on the pH of the aqueous solution, the species of said equilibria also varying in suitability as nucleophile. The following equilibria (4) to (8) are known:

SO₂(gaseous)<->SO₂(dissolved) (4)

SO₂(dissolved)+H₂O<->H₂SO₃ (5)

H₂SO₃<->HSO₃⁻+H⁺ (6)

HSO₃⁻<->SO₃²⁻+H⁺ (7)

2HSO₃⁻<->S₂O₅²⁻+H₂O (8)

Sulfurous acid and its salts are used as preservatives in the food industry, since they exhibit an antimicrobial effect. This means that sulfurous acid and its salts can kill microorganisms, which is attributable to the inactivation of the enzymes necessary for microorganism viability. A person skilled in the art would therefore expect that the CysM enzyme would also be inactivated when using sulfurous acid or its salts and that L-cysteic acid would not be preparable by the process disclosed in EP 1 247 869 B1.

For the aforementioned reasons, it was surprising to a person skilled in the art that, when sulfite and OAS are used in a biotransformation, L-cysteic acid can be produced.

In principle, all conceivable salts of sulfurous acid are suitable for the reaction. Preferably, the process is characterized in that the salt of a sulfurous acid used is Na₂SO₃, K₂SO₃, (NH₄)SO₃, NaHSO₃ (or its anhydride Na₂S₂O₅) or KHSO₃. Particularly preferably, the salt of a sulfurous acid used is Na₂SO₃, NaHSO₃ (or its anhydride Na₂S₂O₅) and (NH₄)₂SO₃ and especially preferably Na₂SO₃ and NaHSO₃ (or its anhydride Na₂S₂O₅).

It is conceivable to use gaseous sulfur dioxide, the anhydride of sulfurous acid, which can be introduced into the reaction batch, where it is hydrated to sulfurous acid H₂SO₃ and, depending on the pH, is in an equilibrium with the deprotonated forms HSO₃⁻ and SO₃²⁻.

The process requires the availability of OAS. Conceivable are chemical processes for producing OAS, for example by acetylation of L-serine, which is costly because of the high prices of L-serine, or else the production of the racemate O-acetyl-D/L-serine, which can be used directly, or OAS is obtained from the racemate beforehand, for example by resolution. In the case of direct acetylation, N-acetyl-L-serine (NAS) may be formed as a by-product, for example by nonselective acetylation on the hydroxyl or amino group of L-serine or the known rearrangement of OAS to NAS at neutral to alkaline pH values (Tai et al. (1995), Biochemistry 34:12311-12322), which reduces yields or requires the prior introduction of a protective group on the amino group of L-serine. The direct acetylation of L-serine is therefore not practical for an economically viable process.

The biotechnological production of OAS is also known, as disclosed in EP 1 233 067 B1 for example. This involves the use of organisms which exhibit deregulated cysteine metabolism and therefore provide a high level of OAS. Cost-effective production systems for producing OAS are available as a result.

In a preferred embodiment, the process is characterized in that OAS is enriched from fermentative production. Fermentative production can be carried out either with the aid of GMOs or with the aid of organisms that are not GMOs.

In a particularly preferred embodiment, the process is characterized in that OAS is produced fermentatively with the aid of microorganisms that are not GMOs, in which case it is especially preferred that OAS is produced fermentatively with the aid of the strain E. coli W3110/pACYC-cysEX-GAPDH-ORF306. The last-mentioned specifically preferred embodiment is disclosed in example 1.

The process of the present invention for producing L-cysteic acid is preferably characterized in that it is a natural production process. This means that not only are no GMOs used in the process, but also both the reactant OAS and the enzyme OAS sulfhydrylase stem from natural production, i.e., are neither produced using GMOs nor produced chemically.

This particularly preferred embodiment is disclosed in the examples according to the invention, which describe a natural production process for L-cysteic acid in which both OAS and the OAS sulfhydrylase CysM are produced naturally. Both the OAS-producing strain E. coli W3110/pACYC-cysEX-GAPDH-ORF306 (example 1) and the CysM production strain E. coli DH5α/pFL145 (example 2) stem from self-cloning and are not classified as GMOs. The fact that both OAS and the OAS sulfhydrylase can be produced without the use of GMOs gives rise to a particular advantage of the invention, since the invention discloses a natural production process for L-cysteic acid, there now being great interest in this owing to the possible applications in the feed sector and in cosmetics.

A person skilled in the art can use isotope analysis to determine whether a substance they wish to use as reactant in the process, such as OAS, stems from chemical or fermentative production. An isotope analysis method capable of differentiating is described for example in Sieper et al., Rapid Commun. Mass Spectrom. (2006) 20:2521-2527 and is based on determination of the isotope ratios for e.g. carbon or nitrogen, which vary according to whether a product stems from chemical (petroleum-based) production or fermentative production (from plant-based raw materials).

An advantage of the present invention is that an OAS-containing fermenter broth, as obtained for example from a fermentation carried out according to EP 1 233 067, can be used directly in the process according to the invention as a source of OAS following removal of the particulate biomass, for example by centrifugation without further workup, purification or isolation steps, including extraction, adsorption, ion exchange chromatography, precipitation and crystallization. This procedure is particularly economical and avoids isolating an unstable compound.

A fermentative process for producing OAS is disclosed in EP 1 233 067 B1 and in example 1 of the present invention, using the strain E. coli W3110/pACYC-cysEX-GAPDH-ORF306. This strain is deposited according to the Budapest Treaty at the DSMZ (German Collection of Microorganisms and Cell Cultures GmbH, Braunschweig) under the number DSM 13495.

The process is preferably characterized in that the OAS sulfhydrylase, including preferably CysM, stems from fermentative production, is particularly preferably produced fermentatively with the aid of microorganisms that are not GMOs, and is especially preferably produced with the aid of an E. coli strain, including especially preferably with the aid of the strain E. coli DH5a/pFL145.

Example 2 discloses procedure for the fermentative biotechnological production of CysM with the strain E. coli DH5a/pFL145. The production strain consists of a host strain, such as E. coli DH5a in this case, and a gene construct suitable for expressing the OAS sulfhydrylase, preferably the gene construct pFL145. Host strain and gene construct, and the production of the production strain, are described in EP 1 247 869 B1 (Wacker). The production strain is deposited according to the Budapest Treaty at the DSMZ-German Collection of Microorganisms and Cell Cultures GmbH (Braunschweig) under the number DSM 14088.

In the process according to the invention, the OAS sulfhydrylase obtained by fermentation can be used either as a fermenter broth without further workup or as a cell suspension after reisolation of the cells from the fermenter broth, for example by centrifugation. Furthermore, the OAS sulfhydrylase can be used in the form of a cell homogenate after mechanical disruption of the cell suspension or in the form of chemically permeabilized cells (e.g., by chloroform) or else as a cell extract after removal of particulate constituents from the cell homogenate or else as an enzyme purified by chromatography, for example.

Preference is given to the use of the OAS sulfhydrylase as a fermenter broth without further workup, as a cell suspension after reisolation of the cells from the fermenter broth or else as a cell homogenate after mechanical disruption of the cell suspension or in the form of chemically permeabilized cells (e.g., by chloroform).

Particular preference is given to the use of the OAS sulfhydrylase as a cell suspension after reisolation of the cells from the fermenter broth or as a cell homogenate.

In an especially preferred embodiment, the process is characterized in that the cells of the production strain that have been isolated from the fermenter broth and resuspended are used as the OAS sulfhydrylase.

In a particularly preferred embodiment, the process for producing L-cysteic acid is characterized in that both the OAS sulfhydrylase and OAS are produced by fermentation.

OAS as the reactant of the biotransformation process according to the invention isomerizes from a pH of approx. pH 7 to N-acetyl-L-serine and is then no longer suitable for reaction with sulfite to form L-cysteic acid. The mechanism of the reaction was studied in Tai et al. (1995), Biochemistry 34:12311-12322 and involves an intramolecular, nucleophilic attack by the deprotonated amino group on the carbonyl carbon of the acyl radical. This reaction is suppressed with decreasing pH, and so the compound is stable at pH 4.0, for example.

The biotransformation process according to the invention is therefore distinguished by the fact that the reaction of OAS to form L-cysteic acid is carried out under pH conditions which minimize the isomerization of OAS to N-acetyl-L-serine.

Preferably, the process is characterized in that the reaction is carried out at a pH value which is at least 5.5 and is ≤7.5, particularly preferably ≤7.0 and especially preferably ≤6.5.

In a further preferred embodiment of the biotransformation process, the substrate OAS is metered into the reaction batch composed of OAS sulfhydrylase and sulfite in a so-called feed process (example 5). In the OAS metered in, a pH which suppresses the isomerization to N-acetyl-L-serine is set, preferably a pH≤6.5, particularly preferably a pH≤6.0 and especially preferably a pH≤5.5. At the same time, the pH in the reaction batch is adjusted in such a way that it promotes the reaction to form L-cysteic acid.

According to equation (2), the reaction of OAS to form L-cysteic acid releases stoichiometric amounts of acetic acid, which may lead to a decrease in pH in the batch as the reaction proceeds. Since an excessively low pH affects the activity of the OAS sulfhydrylase, it is necessary to prevent an excessively large drop in pH. This can be done passively by a suitable highly concentrated buffer in the batch or can be achieved actively by a measurement and control unit.

Preference is given to active pH control by a measurement and control unit, as disclosed in example 5, which, in the event of deviation of the pH from the target value, restores the desired pH by metered addition of an alkaline solution or acid (so-called pH-stat method).

The reaction temperature is preferably chosen between 5° C. and 70° C. Preference is given to a reaction temperature between 10° C. and 60° C., particular preference to between 15° C. and 50° C., and especial preference to between 20° C. and 40° C.

The process for producing L-cysteic acid is preferably carried out in an aqueous environment, i.e., the solvent used for the reaction is preferably water.

The process according to the invention for producing L-cysteic acid can be carried out in discontinuous operation or continuous operation. In discontinuous operation (batch operation), all reactants are added to the batch in the course of the reaction and the batch is worked up after the reaction has ended. In continuous operation, OAS, OAS sulfhydrylase and a salt of sulfurous acid are constantly metered in during the reaction and a solution containing the product L-cysteic acid is simultaneously removed from the batch. What is established is a steady state in which the reactants are metered in in such a way that they can completely react to form the product L-cysteic acid during the residence time in the reaction vessel. A process for continuous production of unnatural amino acids is disclosed in, for example, EP 1 247 869 B1 (Wacker).

Preference is given to discontinuous operation of the process according to the invention for producing L-cysteic acid.

Preferably, the process is characterized in that the concentration of the salt of sulfurous acid is at least in equimolar concentration, particularly preferably in at least 1.5-fold molar excess, especially preferably in at least 2-fold molar excess and additionally preferably in at least 5-fold molar excess to OAS.

The OAS concentration in the batch is preferably at least 1 g/L, particularly preferably at least 10 g/L, and especially preferably at least 40 g/L.

In the biotransformation of OAS, the molar yield of L-cysteic acid based on the molar amount of OAS used is preferably at least 60%, particularly preferably at least 70% and especially preferably at least 80%.

The invention will be further illustrated by the following examples without being restricted by them:

### EXAMPLE 1: PRODUCTION OF OAS

The strain E. coli W3110/pACYC-cysEX-GAPDH-ORF306 disclosed in EP 1 233 067 B1 (Wacker) and deposited according to the Budapest Treaty at the DSMZ-German Collection of Microorganisms and Cell Cultures GmbH (Braunschweig) under the number DSM 13495 was used. OAS was produced by fermentation as described in EP 1 233 067 B1. At the end of fermentation, OAS was stabilized by setting a pH of 4.5 using 21% (v/v) phosphoric acid. The cells were removed by centrifugation at 4000 rpm for 10 min (Heraeus Megafuge 1.0 R). The HPLC-determined content of OAS in the fermentation supernatant was 15.3 g/L.

### HPLC Analysis of OAS and L-Cysteic Acid:

For quantitative determination of the compounds analyzed in the examples, an HPLC method calibrated respectively for OAS and L-cysteic acid was employed; all reference substances used for calibration were commercially available (Sigma-Aldrich). An Agilent 1260 Infinity II HPLC system was used, which was equipped with a unit from the same manufacturer for pre-column derivatization with o-phthaldialdehyde (OPA derivatization) as is known from the analysis of amino acids. For detection of the OPA-derivatized products OAS and L-cysteic acid, the HPLC system was equipped with a fluorescence detector. The detector was set to an excitation wavelength of 330 nm and an emission wavelength of 450 nm. Also used were an Accucore™ aQ column from Thermo Scientific™, length 100 mM, internal diameter 4.6 mm, particle size 2.6 μm, thermally equilibrated at 40° C. in a column oven.

Eluent A: 25 mM Na phosphate, pH 6.0. Eluent B: methanol. The separation was carried out in gradient mode: 10% eluent B to 60% eluent B over 0-25 min, followed by 60% eluent B to 100% eluent B over 2 min, followed by 100% eluent B for a further 2 min, at a flow rate of 0.5 ml/min. Retention time of L-cysteic acid: 3.2 min. Retention time of OAS: 17.0 min.

### EXAMPLE 2: PRODUCTION OF THE ENZYME CYSM

The strain E. coli DH5a/pFL145 disclosed in EP 1 247 869 B1 (Wacker) and deposited according to the Budapest Treaty at the DSMZ-German Collection of Microorganisms and Cell Cultures GmbH (Braunschweig) under the number DSM 14088 was used. CysM enzyme was produced both by growth in a shake flask and by fermentation.
A) Growth in a shake flask: A preculture of the strain E. coli DH5α/pFL145 was prepared in LBamp medium (10 g/l tryptone (GIBCO™), 5 g/l yeast extract (BD Biosciences), 5 g/l NaCl, 100 mg/L ampicillin (Sigma-Aldrich)) (growth at 37° C. and 120 rpm overnight). 25 ml of preculture were used as inoculum for a main culture of 250 ml of LBamp medium (1 L Erlenmeyer flask with baffles). The main culture was shaken at 30° C. and 110 rpm. After 4 h, a cell density OD₆₀₀ of 1.0/ml was reached (OD₆₀₀: photometric determination of cell density per ml of cell suspension by determination of absorbance at 600 nm; Genesys™ 10S spectrophotometer UV-Vis from Thermo Scientific™). Then the inducer tetracycline (Sigma-Aldrich, 3 mg/L final concentration) was added and growth was continued for another 20 h at 30° C. and 110 rpm. At the end of growth, the cell density OD₆₀₀ was 3/ml.
B) Fermentative production of CysM with the strain E. coli DH5α/pFL145 is disclosed in EP 1 247 869 B1. The cells from the fermentation were removed by centrifugation at 4000 rpm for 10 min (Heraeus Megafuge 1.0 R) and suspended in KPi6.5 buffer (0.1 M K phosphate, pH 6.5), so that the cell density OD₆₀₀ was 90/ml.

The cells from the shake flask growth or fermentation were isolated by centrifugation (10 min at 15000 rpm, Sorvall RC5C centrifuge, equipped with an SS34 rotor) for further use. For the further use for production of a cell homogenate, as described below, the cell pellet was resuspended in KPi6.5 buffer as a cell suspension. The cell suspension was prepared by using an amount of KPi6.5 buffer sufficient for the cell density OD₆₀₀ to be 30/ml: for example, 50 ml of cells from the shake flask growth having an OD₆₀₀ of 3/ml were centrifuged and resuspended in 5 ml of KPi6.5 buffer (10-fold concentration) or 1 ml of cells from the fermentation having an OD₆₀₀ of 90/ml were resuspended in 3 ml of KPi6.5 buffer (3-fold dilution).

This produced the cells of the strain E. coli DH5α/pFL145, isolated from the fermenter broth and resuspended, that were used in the following as OAS sulfhydrylase CysM in the process according to the invention.

To prepare a cell homogenate, the FastPrep-24™ 5G cell homogenizer from MP Biomedicals was used. 1 ml of cell suspension in KPi6.5 buffer having a cell density OD₆₀₀ of 30/ml was disrupted in manufacturer-assembled 1.5 ml tubes containing glass beads (“Lysing Matrix B”) (3×20 sec at a shaking frequency of 6000 rpm with a 30 sec pause each time between the intervals). The cell homogenate obtained was used directly as OAS sulfhydrylase (CysM enzyme) in the process according to the invention or used for preparation of a cell extract.

To prepare a cell extract, the cell homogenate obtained was centrifuged (15 000 rpm for 10 min, Sorvall RC5C centrifuge, equipped with an SS34 rotor), and the supernatant designated cell extract and used as OAS sulfhydrylase (CysM enzyme) in the process according to the invention or used further for determination of CysM enzyme activity.

The protein content of the cell extract was determined by means of a Qubit 3.0 Fluorometer from Thermo Fisher Scientific using the “Qubit@ Protein Assay Kit” according to the manufacturer's instructions. The protein content of the cell extract from the shake flask growth was 5.3 mg/ml. The protein content of the cell extract from the fermentation was 4.0 mg/ml.

CysM enzyme activity was determined as described in EP 1 247 869 B1 (Wacker). To this end, OAS (Sigma-Aldrich) was incubated at 37° C. in the presence of Na S and cell extract from the growth of the strain E. coli DH5α/pFL145. The assay (0.4 ml final volume) in KPi6.5 buffer contained 10 mM OAS (addition from a 200 mM stock solution in 500 mM sodium succinate buffer pH 5.5), 10 mM sodium sulfide Na₂S and 5 μl of CysM-containing cell extract. The cysteine produced in the CysM reaction was determined using ninhydrin (Sigma-Aldrich) according to the method by Gaitonde (1967), Biochem. J. 104:627-633. The CysM enzyme activity in the cell extract from the growth of the strain E. coli DH5α/pFL145 in a shake flask was 57.1 U/ml. Since the cells from the shake flask growth (OD₆₀₀ of 3/ml) had been concentrated 10-fold for the preparation of the cell extract, the enzyme activity in cells from the shake flask growth was 5.7 U/ml. The CysM enzyme activity in the cell extract following fermentation of the strain E. coli DH5α/pFL145 was 58.1 U/ml. Since the cells from the fermentation (OD₆₀₀ of 90/ml) had been diluted to an OD₆₀₀ of 30/ml for preparation of the cell extract, the enzyme activity in the concentrated (OD₆₀₀ of 90/ml) cell suspension of the fermenter cells was 174.4 U/ml.

The specific CysM enzyme activity of the cell extract from the growth of the strain E. coli DH5α/pFL145 in a shake flask was 10.8 U per mg of protein. The specific CysM enzyme activity of the cell extract following fermentation of the strain E. coli DH5α/pFL145 was 14.5 U/mg. Assuming that CysM activity was completely released from the cells during the preparation of the cell extract, the CysM enzyme activity determined in the cell extracts was equated to the enzyme activity present in CysM cell suspensions in the following examples.

1 U/ml CysM enzyme activity is defined as the production of 1 μmol of cysteine per min from OAS and Na₂S under assay conditions in 1 ml of cell extract (volume activity). Specific CysM enzyme activity in U per mg of protein is obtained by dividing the volume activity of the cell extract (U/ml) by the protein concentration of the cell extract (mg/ml) and is defined as CysM enzyme activity in U based on 1 mg of protein in the cell extract.

### EXAMPLE 3: PRODUCTION OF L-CYSTEIC ACID FROM COMMERCIALLY AVAILABLE OAS AND NA₂SO₃ WITH THE AID OF CYSM PRODUCED IN A SHAKE FLASK CULTURE

Two Batches were Carried Out in Parallel:

Batch 1: A 100 ml Erlenmeyer flask was initially charged with 8.25 ml of NaPi6.5 buffer (50 mM Na phosphate, pH 6.5), and added in succession were 1 ml of a 0.2 M solution of Na₂SO₃ in NaPi6.5 buffer, 0.4 ml of CysM cell extract from the shake flask growth (from example 2A) having an activity of 57.1 U/ml (2.3 U/ml final concentration in the batch) and 350 μl of a 0.2 M solution of OAS×HCl (Sigma-Aldrich) in 0.5 M Na succinate, pH 5.5. The batch volume was 10 ml.

Batch 2: The batch (comparative batch without Na₂SO₃) had the same composition as batch 1. Instead of the Na₂SO₃ solution, batch 2 received 1 ml of NaPi6.5 buffer.

Both batches were incubated at 37° C. and 140 rpm in a chest shaker (Infors). After 1 h and 3 h, 1 ml of the batches in each case were incubated at 80° C. for 5 min to stop the reaction and centrifuged, and the supernatant was analyzed by HPLC. The amount of L-cysteic acid detected by HPLC is shown in Table 1.

TABLE 1
HPLC-detected amount of L-cysteic acid according
to reaction time, using commercially available OAS and
Na₂SO₃ and a CysM-containing cell extract.

| | Batch 1 with Na₂SO₃ | Batch 2 without Na₂SO₃ |
|---|---|---|
| Time [h] | L-cysteic acid [mg/L] | L-cysteic acid [mg/L] |
| 0 | 0.0 | 0.0 |
| 1 | 78.0 | 0.0 |
| 3 | 95.8 | 0.0 |

### EXAMPLE 4: PRODUCTION OF L-CYSTEIC ACID FROM OAS-CONTAINING CULTURE SUPERNATANT FROM FERMENTATION AND NA₂SO₃ WITH THE AID OF CYSM PRODUCED IN A SHAKE FLASK CULTURE

A 100 ml Erlenmeyer flask was initially charged with 1 ml of cell culture supernatant from the fermentation of the strain E. coli W3110/pACYC-cysEX-GAPDH-ORF306 having an OAS content of 15.3 g/L (from example 1), and added in succession were 6 ml of NaPi6.5 buffer, 1 ml of a 1 M solution of Na₂SO₃ in NaPi6.5 buffer and 2 ml of CysM cell suspension from the shake flask growth (from example 2A, cell density OD₆₀₀ of 30/ml; 57.1 U/ml of CysM enzyme activity). The batch volume was 10 ml. The CysM enzyme activity in the batch was 11.4 U/ml. The batch was incubated at 37° C. and 140 rpm in a chest shaker (Infors). After 2 h, 1 ml of the batch was incubated at 80° C. for 5 min and centrifuged and the supernatant was analyzed by HPLC for the content of OAS and L-cysteic acid. The course of the reaction over time is summarized in Table 2.

TABLE 2
HPLC-detected amount of L-cysteic acid and OAS,
using an OAS-containing cell culture supernatant, Na₂SO₃
and a CysM-containing cell suspension.

| Time [h] | OAS [mg/L] | L-cysteic acid [mg/L] |
|---|---|---|
| 0 | 1530.0 | 0.0 |
| 2 | 0.0 | 1473.3 |

### EXAMPLE 5: PREPARATIVE PRODUCTION OF L-CYSTEIC ACID BY BIOTRANSFORMATION OF OAS AT CONSTANT PH

A 0.5 L thermostatable double-walled glass vessel (Diehm) was connected to a thermostat (Lauda) via a hose connection and adjusted to a temperature of 37° C.

50 ml of CysM-containing cell suspension in KPi6.5 buffer (OD₆₀₀ of 90/ml, 8720 U of CysM enzyme activity) from the fermentation of the strain DH5α/pFL145 (from example 2B) and 6.6 ml of a 400 g/L solution of Na₂S₂O₅ (13.9 mmol, molecular weight of 190.1 g/mol) in KPi6.5 buffer were initially charged. In dissolved form, this corresponded to 27.8 mmol of NaHSO3 (1.78-fold molar excess to the OAS amount of 15.6 mmol that is metered in later). The batch was stirred with a magnetic stirrer. The batch was also equipped with a pH electrode (Mettler Toledo), which was connected to a pH control unit (TitroLine alpha titrator, Schott) which was operated in pH-stat mode according to the manufacturer's instructions. Under pH-stat conditions, the pH in the reaction vessel was kept constant at the set pH of 6.5 over the entire duration of the reaction by metered addition of 2 M NaOH from a burette connected to the control unit. 150 ml of OAS-containing cell culture supernatant (OAS content: 15.3 g/L, 2.3 g; 15.64 mmol) from the fermentation of the strain E. coli W3110/pACYC-cysEX-GAPDH-ORF306 (example 1) were metered into the batch from a reservoir via a pump (Watson Marlow 101U/R peristaltic pump) at a flow rate of 0.35 ml/min.

The reaction time was 19 h. Since the batch was carried out in an open reaction vessel, the batch volume was 185 ml after completion of the reaction owing to evaporation. 0.5 h, 3 h and 19 h after the start of the reaction, a 1 ml aliquot of the batch was removed in each case and the content of L-cysteic acid was analyzed by HPLC. The formation of L-cysteic acid over time is summarized in Table 3. After a 19 h reaction time, the L-cysteic acid content in the batch was 12 970 mg/L (76.65 mM), which corresponded to an absolute molar yield of 14.18 mmol of L-cysteic acid for a batch volume of 185 ml. Based on the amount of OAS used of 15.64 mmol, this corresponded to a yield of 90.1%.

TABLE 3
HPLC-detected amount of L-cysteic acid according to reaction
time, using an OAS-containing fermentation supernatant, NaHSO₃
and a cell suspension of CysM-containing fermenter cells

| Time [h] | L-cysteic acid [mg/L] | L-cysteic acid [mM] |
|---|---|---|
| 0.5 | 758.0 | 4.47 |
| 3 | 4244.0 | 25.08 |
| 19 | 12970.0 | 76.65 |

1-15. (canceled)

16. A process for producing L-cysteic acid, comprising:
wherein O-acetyl-L-serine (OAS) is converted using at least one enzyme selected from the class of O-acetyl-L-serine sulfhydrylases (OAS sulfhydrylases, EC 4.2.99.8) in the presence of a salt of sulfurous acid, wherein the OAS sulfhydrylase is CysM and the biotransformation is carried out under active pH control and the OAS concentration in the batch is at least 10 g/L.

17. The process of claim 16, wherein the OAS sulfhydrylase is a bacterial enzyme.

18. The process of claim 16, wherein the OAS sulfhydrylase is CysM from the strain E. coli.

19. The process of claim 16, wherein the OAS sulfhydrylase stems from fermentative production.

20. The process of claim 16, wherein the OAS sulfhydrylase is produced fermentatively with the aid of microorganisms that are not genetically modified organisms (GMOs).

21. The process of claim 16, wherein the OAS sulfhydrylase is produced fermentatively with the aid of the strain E. coli DH5α/pFL145.

22. The process of claim 16, wherein the OAS stems from fermentative production.

23. The process of claim 16, wherein the OAS is produced fermentatively with the aid of microorganisms that are not GMOs.

24. The process of claim 16, wherein the OAS is produced fermentatively with the aid of the strain E. coli W3110/pACYC-cysEX-GAPDH-ORF306.

25. The process of claim 16, wherein the process is a natural production process, a natural production process being defined by the fact that no genetically modified organisms (GMOs) are used in the process and the reactant OAS and the enzyme OAS sulfhydrylase stem from natural production, i.e., are not produced using GMOs and are not produced chemically.

26. The process of claim 16, wherein the salt of a sulfurous acid used is Na₂SO₃, K₂SO₃, (NH₄)₂SO₃, NaHSO₃ (or its anhydride Na₂S₂O₅) or KHSO₃.

27. The process of claim 16, wherein the concentration of the salt of sulfurous acid is at least in equimolar concentration to OAS.

28. The process of claim 16, wherein the reaction is carried out at a pH of at least 5.5 and of less than 7.5.

29. The process of claim 16, wherein the L-cysteic acid is enriched from the reaction batch.
